(19) United States
(12) Patent Application Publication
(10) Pub. No.: US 2024/0337659 A1
(43) Pub. Date: Oct. 10, 2024

(54) METHOD OF DETECTING PROTEINS IN HUMAN SAMPLES AND USES OF SUCH METHODS

(71) Applicant: ProteoMedix AG, Schlieren (CH)

(72) Inventors: Ralph SCHIESS, Zürich (CH); Alcibiade ATHANASIOU, Dättwil (CH); Ramy HUBER, Wettingen (CH); Anja WITTIG, Berikon (CH)

(73) Assignee: ProteoMedix AG, Schlieren (CH)

(21) Appl. No.: 18/574,572

(22) PCT Filed: Jun. 16, 2022
(86) PCT No.: PCT/EP2022/066456

§ 371 Date: Dec. 27, 2023

(30) Foreign Application Priority Data
Jun. 29, 2021 (CH) 00752/21

(51) Int. Cl. G01N 33/574; G01N 33/68

(52) U.S. Cl. CPC G01N 33/57434; G01N 33/6893; G01N 2333/4703; G01N 2333/78; G01N 2333/96411; G01N 2333/96419; G01N 2470/04

(57) ABSTRACT

A method for collecting information about the health status of a subject involving the quantitative detection, in serum, plasma or blood of the subject, of the concentration of at least four of the systems selected from the group consisting of: THBS1, LUM, FN1, LG3BP, MMP9, as well as PSA.

### TECHNICAL FIELD

The present invention relates to the field of methods for the measurement of proteins in human samples, in particular in human serum, plasma or blood, and it also relates to assays and uses of such assays, in particular for risk assessment.

### PRIOR ART

The measurement of proteins in human samples of a person is a powerful tool for the supervision and the risk assessment of the general status of the person, in particular as concerns the nutritional and health status of the person.

Prostate cancer (PCa) is the most frequently diagnosed cancer in men and the second leading cause of male cancer related deaths in the US.

The diagnosis and treatment of PCa, despite decennial research efforts, are still a major challenge in the clinics. PCa progression is unfortunately silent, and an early detection of faster progressing and potentially dangerous lesions is crucial for the patient's health, since complete remission and cure from the disease is possible only at early stages of the disease.

The most frequently used noninvasive test for PCa detection relies on the measurement of the Prostate Specific Antigen (PSA) in the blood coupled with digital rectal examination (DRE). PSA is a protein produced by the epithelial cells of the prostate gland. PSA is also known as kallikrein III, seminin, semenogelase, γ-seminoprotein and P-30 antigen and it is a 34 kD glycoprotein present in small quantities in the serum, plasma or blood of normal men, and is often elevated in the presence of PCa and in other prostate disorders. A blood test to measure PSA coupled with DRE is the most effective test currently available for the early detection of PCa. Higher-than-normal levels of PSA are associated with both localized and metastatic PCa.

The diagnostic accuracy of PSA alone is only around 60% and the methodology has major drawbacks in specificity (too many false positive cases that undergo unneeded prostate biopsy or surgery). Indeed, PSA levels can also be increased by prostate infection, irritation, benign prostatic hypertrophy (enlargement) or hyperplasia (BPH), and recent ejaculation, producing a false positive result.

Thus, PCa diagnosis is currently hampered by the high false-positive rate of PSA evaluations, which consequently may lead to a high number of prostate biopsies with negative diagnostic findings. Further, these unnecessary biopsies can have potential side effects. Recent recommendations against widespread screening of men for PCa using PSA have resulted in fewer men being screened for PCa, and fewer early-stage cases being detected.

A reliable and non-invasive diagnostic/prognostic procedure that is avoiding false positive and false negative results is thus still lacking, even though novel methodologies based on the simultaneous measurement of various parameters (e.g. free and total PSA) are emerging as tools to increase the overall diagnostic accuracy. Most PSA in the blood is bound to proteins. A small amount is not protein bound and is called free PSA. In men with prostate cancer the ratio of free (unbound) PSA to total PSA is decreased. The risk of cancer increases if the ratio of free to total PSA (% fPSA) is less than 25%. The lower the ratio, the greater the probability of PCa. However, both total and free PSA increase immediately after ejaculation, returning slowly to baseline levels within 24 hours, and also other mechanisms not related to PCa can influence the free to total PSA ratio.

New diagnostic tools, ideally non-invasive ones, are urgently needed to improve PCa diagnosis and reduce unnecessary biopsies and overtreatment. More accurate diagnostics from easily accessible sample types like blood will allow physicians and patients to make more informed decisions about potential cases of PCa and whether a prostate biopsy is required.

Similar to diagnosis, treatment and/or prognosis of PCa remains a major challenge due to heterogeneity of the disease. Although multiple mechanisms of PCa have been suggested, the lack of suitable signatures able to stratify patients and key target proteins for therapeutic intervention cures are still not within reach.

One approach to find a suitable diagnostic system for prostate cancer is proposed in WO 2009/138392, where it is proposed to measure at least two of a list of 24 proteins known to be present in human blood, and expected to be down-regulated or up-regulated depending on the health status of the corresponding patient.

The problem with known approaches is that they still suffer a lack of sensitivity and in particular specificity in terms of which cancer is actually present, and a lack of diagnostic reliability in terms of avoiding false positive and false negative results. A further problem is the actual availability of corresponding detection probes, be it antibody-based or any other type of detection, making the corresponding tools suitable not only for academic purposes but also for broad applications. A further issue is that the corresponding detection systems should be simple and not entail a large number of individual measurements.

WO-A-2018011212 proposes a method for collecting information about the health status of a subject involving the quantitative detection, in serum, plasma or blood of the subject, of the concentration of THBS1, the proportion of free PSA (% fPSA), preferably including the concentration of at least one protein selected from the group consisting of CTSD, OLFM4, ICAM1.

PCa can be managed through curative therapies, such as Radical Prostatectomy (RP), which provides excellent cancer control of localized PCa. Approximately 30% of surgically treated men will experience biochemical recurrence (BCR), being at significant risk for clinical cancer progression (metastases) and have the need for institution of systemic therapy.

Clinical stage, pretreatment PSA levels and prostate biopsy Gleason grade have been shown to be reliable and independent predictors of treatment failure. Clinical risk profiles (pre-treatment nomograms (e.g. Kattan or CAPRA score) were designed to identify patients who can safely avoid aggressive therapy or to select potential candidates for neoadjuvant clinical trials.

The usefulness of current models, however, depends on their predictive accuracy. Preoperative PSA levels may reflect primarily benign prostate hyperplasia (BPH) rather than the presence of PCa in populations in which PSA is regularly used for screening. After biopsy, the prediction for aggressiveness of PCa is difficult even with the use of nomograms incorporating clinical information in their algorithm. This applies especially for patients with a low and very-low risk of disease progression, for whom active surveillance is becoming a widely adopted strategy.

US-A-2020292548 discloses methods for diagnosing the presence of biochemical recurrence (BCR) in prostate cancer in a subject, such methods including the detection of levels of a variety of biomarkers diagnostic of BCR. Compositions in the form of kits and panels of reagents for detecting the biomarkers of the invention are also provided.

Oguic et al in Patholog Res Int. 2014; 2014:26219 evaluated the expression of matrix metalloproteinase 2 (MMP-2) and matrix metalloproteinase 9 (MMP-9) in prostate cancer in the main tumor mass and tumor cells at the positive margin as well as the influence of these biomarkers on the biochemical recurrence of the disease in prostatectomy patients. Tissue microarrays of 120 archival prostate carcinoma samples were immunohistochemically evaluated for MMP-2 and MMP-9 expression and compared with clinicopathological parameters. Tumors with positive surgical margins showed significantly higher overall expression of MMP-9 versus tumors with negative resection margins (P=0.0121). MMP-9 expression was significantly elevated in tumors from patients who had biochemical recurrence (P=0.0207). In the group of patients with negative margins, MMP-9 expression above the cut-off value was significantly associated with recurrence (P=0.0065).

Multivariate analysis indicated that MMP-9 is a good predictor of biochemical recurrence (odds ratio=10.29; P=0.0052). Expression of MMP-2 in tumor cells was significantly higher at the positive margins than in the main tumor mass (P=0.0301). The results highlight the potential value of MMP-2 and MMP-9 expression for predicting the behavior of prostate tumors after prostatectomy with both positive and negative surgical margins.

Miyata et al in Prostate. 2015 January;75(1):84-91 report, that neoadjuvant hormonal therapy (NHT) is performed to improve the outcome in organ-confined prostate cancer. However, there is little information regarding the relationship between angiogenesis and NHT. The aim of this study was to identify a suitable method to evaluate the angiogenic status of tissue, and to determine the prognostic value of this method for biochemical recurrence in patients who had undergone radical prostatectomy after NHT. They analyzed 108 formalin-fixed specimens from patients treated by radical prostatectomy. NHT was administered in 48 patients (52.9%) and 60 patients who had a similar Gleason score and pT stage were selected as a non-NHT treated control group. Microvessel density (MVD) was measured using anti-CD31, anti-CD34, and anti-CD105 antibodies. The expressions of vascular endothelial growth factor (VEGF)-A and thrombospondin (TSP)-1 were also evaluated by immunohistochemistry. The prognostic value of CD31-, CD34-, and CD105-MVD for biochemical recurrence was investigated. The mean/SD of CD105-MVD in the NHT group (13.3/4.7) was reported to be significantly (P<0.001) lower than that in the non-NHT group (125.8/7.3). In the NHT group, CD105-MVD was associated with pT stage and it was positively correlated with VEGF-A expression (r=0.56, P<0.001) and negatively correlated with TSP-1 expression (r=0.42, P=0.003). CD105-MVD was identified as a significant predictor of biochemical recurrence (BCR) in patients treated with NHT (log rank test, P<0.001). Although CD31-and CD34-MVD were significantly associated with pT stage or Gleason score in non-NHT group, they were not associated with pathological features and BCR in NHT group. Their results indicate that CD105-MVD reflects the angiogenic conditions in prostate cancer tissues treated with NHT. CD105-MVD was also identified as a significant and independent predictor of biochemical recurrence in prostate cancer patients who underwent radical prostatectomy with NHT.

Coulsen-Thomas et al in Experimental Cell Research 319 (7) report, that the stromal reaction surrounding tumors leads to the formation of a tumor-specific microenvironment, which may play either a restrictive role or a supportive role in the growth and progression of the tumors. Lumican, a small leucine-rich proteoglycan (SLRP) of the extracellular matrix (ECM), regulates collagen fibrillogenesis. Recently, lumican has also been shown to regulate cell behavior during embryonic development, tissue repair and tumor progression.

The role of lumican in cancer varies according to the type of tumor. In this study they analyze the role of lumican in the pathogenesis of prostate cancer both in vivo and in vitro. Overall lumican up-regulation was observed in the primary tumors analyzed through both real-time PCR and immunostaining. The increase in lumican expression was observed in the reactive stroma surrounding prostate primary tumors with fibrotic deposition surrounding the acinar glands. In vitro analysis demonstrated that lumican inhibited both the migration and invasion of metastatic prostate cancer cells isolated from lymph node, bone and brain. Moreover, prostate cancer cells seeded on lumican presented a decrease in the formation of cellular projections, lamellipodia detected by a decreased rearrangement in ZO-1, keratin 8/18, integrin β1 and MT1-MMP, and invadopodia detected by disruption of α-smooth muscle actin, cortactin and N-WASP. Moreover, a significant increase in prostate cancer cell invasion was observed through the peritoneum of lumican knockout mice, further demonstrating the restrictive role lumican present in the ECM has on prostate cancer invasion. In conclusion, lumican present in the reactive stroma surrounding prostate primary tumors plays a restrictive role on cancer progression, and we therefore postulate that lumican could be a valuable marker in prostate cancer staging.

Behnsawy et al in BJU Int. 2013 January;111(1):30-37 report the aim to analyse the expression patterns of multiple molecular markers implicated in epithelial-mesenchymal transition (EMT) in localized prostate cancer (PC), in order to clarify the significance of these markers in patients undergoing radical prostatectomy (RP). Expression levels of 13 EMT markers, namely E-cadherin, N-cadherin, β-catenin, γ-catenin, fibronectin, matrix metalloproteinase (MMP) 2, MMP-9, Slug, Snail, Twist, vimentin, ZEB1 and ZEB2, in RP specimens from 197 consecutive patients with localized PC were evaluated by immunohistochemical staining.

Of the 13 markers, expression levels of E-cadherin, Snail, Twist and vimentin were closely associated with several conventional prognostic factors. Univariate analysis identified these four EMT markers as significant predictors for biochemical recurrence (BR), while serum prostate-specific antigen, Gleason score, seminal vesicle invasion (SVI), surgical margin status (SMS) and tumour volume were also significant. Of these significant factors, expression levels of Twist and vimentin, SVI and SMS appeared to be independently related to BR on multivariate analysis. There were significant differences in BR-free survival according to positive numbers of these four independent factors. That is, BR occurred in four of 90 patients who were negative for risk factors (4.4%), 21 of 83 positive for one or two risk factors (25.3%) and 19 of 24 positive for three or four risk factors (79.2%).

Measurement of expression levels of potential EMT markers, particularly Twist and vimentin, in RP specimens, in addition to conventional prognostic parameters, are stated would contribute to the accurate prediction of the biochemical outcome in patients with localized PC following RP.

Bair et al. in Prostate. 2006 Feb. 15;66(3):283-93 report 90K/Mac-2 binding protein to be a cell adhesive protein whose level of expression has been correlated with metastatic potential in many different tumor types. The purpose of this investigation was to examine 90K expression in prostate cancer and to determine a possible role for 90K in cancer progression. 90K expression in prostate cell lines and tissue samples was evaluated by immunohistochemistry. Expression in cell lines was also evaluated by Western blot analysis and real-time RT-PCR. Induction of promatrilysin by 90K was evaluated by ELISA. Some of the human prostate cell lines studied expressed 90K. 90K was over-expressed in 38.8% of prostate cancer tumor samples, 7.14% of PIN lesions, and 18.6% of normal tissue. 90K was also shown to induce promatrilysin expression in the prostate cell line, LNCaP. These data demonstrate that 90K is over-expressed in a large fraction of malignant tumors. The fact that 90K can induce expression of promatrilysin indicates a possible role for 90K in cancer progression and metastasis. This suggests that 90K over-expression may be a useful marker for examining prostate cancer progression.

WO-A-2018011212 discloses a method for collecting information about the health status of a subject is proposed involving the quantitative detection, in serum, plasma or blood of the subject, of the concentration of THBS1, the proportion of free PSA (% fPSA), preferably including the concentration of at least one protein selected from the group consisting of CTSD, OLFM4, ICAM1.

### SUMMARY OF THE INVENTION

Therefore, there is a compelling need to identify novel markers that are specifically linked to the presence of biologically aggressive PCa for improved prediction of outcome in populations with moderately elevated PSA levels. Because of these current limitations with models based primarily on total PSA levels and standard pathological tumor grading, we investigated alternative PCa related biomarkers and their association to BCR and adverse pathology (AP) in patients with clinically localized PCa.

We performed univariate and multivariate analysis of multiple protein biomarkers originally discovered in the context of PTEN-mutation using mouse model and proteomics technology. The clinical performance of the combination of multiple biomarkers together with the clinical grade group (GG) and PSA were evaluated for the prediction of BCR after RP compared to the Cancer of the Prostate Risk Assessment (CAPRA) score. In addition, the association with AP was investigated as well.

So the objective was to determine the prognostic utility of a new biomarker combination in PCa patients undergoing Radical Prostatectomy (RP).

Serum samples and clinical data of 557 men who underwent RP for PCa with clinical stage (cT) <3 at Martini Clinic (Hamburg, Germany) were used for analysis. GG was determined using biopsy samples while tumor marker concentrations were measured in serum using immunoassays. The prognostic utility of the proposed marker combination was assessed using Cox proportional hazard regression and Kaplan-Meier analysis. The performance was compared to the CAPRA score in the overall cohort and in a low-risk patient subset.

A multivariable model comprising fibronectin 1 (FN1), galectin-3-binding protein (LG3BP), lumican (LUM), matrix metalloprotease 9 (MMP9), thrombospondin-1 (THBS1) and PSA together with GG was created. The proposed model was a significant predictor of BCR (HR 1.28 per 5 units score, 95% Cl 1.19-1.38, p<0.001). The Kaplan-Meier analysis showed that the proposed model had a better prediction for low-risk disease after RP compared to the CAPRA score (respectively 4.9% vs. 9.1% chance of BCR). In a pre-defined low risk population subset, the risk of BCR using the proposed model was below 5.5% and thus lower when compared to CAPRA score=0-2 (9%), GG<2 (7%) and NCCN=low-risk (6%) subsets. Additionally, the proposed model could significantly (p<0.001) discriminate patients with AP events at RP from those without.

The proposed model is thus unexpectedly and significantly superior to the CAPRA score for the prediction of BCR after RP in the overall cohort as well as a in a pre-defined low risk patient population subset. It is also significantly associated with AP at RP.

More generally speaking, the present invention relates to a method as claimed in the appended claims.

What is claimed and described is a method for collecting information about the health status of a subject involving the quantitative detection, in serum, plasma or blood of the subject, of the concentration of at least four of the systems selected from the group consisting of: THBS1, LUM, FN1, LG3BP, MMP9, as well as (total) PSA.

According to a first preferred embodiment, the method involves the quantitative detection, in serum, plasma or blood of the subject, of the concentration of each of THBS1, LUM, FN1, LG3BP, MMP9, as well as PSA.

Particularly preferably, further the Gleason grade (GG) of at least one preceding biopsy is taken account of, expressed as integer in the range of 1-5.

According to a further preferred embodiment, the proposed method includes:
a first step being performed by contacting the subject's serum, plasma or blood, preferably after dilution thereof, with at least one, preferably two, affinity reagent(s) for each protein and detecting whether binding occurs between the respective protein and the at least one affinity reagent and using quantitative readout of the respective protein's concentration, allowing the calculation of the respective concentration in the original serum, plasma or blood;
a second step of calculating, based on all the protein concentrations as well as the PSA concentration determined in the first step, a combined score value.

After the second step in a third step the risk of BCR after surgery of PCa and/or of AP of the subject can be determined based on the combined score value as determined in the second step, wherein surpassing a corresponding threshold value of the combined score value is taken as positive BCR after surgery and/or as necessity of prostatectomy. Preferably, the combined score value is calculated based on the measured concentrations x_{tPSA}, x_{MMP9}, x_{LG3BP}, x_{THBS1}, x_{FN1}, x_{LUM} and/or the Gleason grade (GG) of at least one preceding biopsy expressed as integer in the range of 1-5 using the following formula:

$$[ 1 - \frac{1}{1 + {e}^{( {β}_{0} + {β}_{1} * {x}_{1} + … + {β}_{k} * {x}_{k} )}} ] * 100$$

and β₀; β_{tPSA}; β_{GG}; β_{MMP9}; β_{LG3BP}; β_{THBS1}; β_{FN1}; β_{LUM}.

Preferably the parameters are chosen as follows, wherein at least one or a combination of the given values are possible
β₀ is in the range of (−2)-0, preferably in the range of (−1.5)-(−0.5);
β_{tPSA} is in the range of 0-0.4, preferably in the range of 0.01-0.31;
β_{GG} in the range of 0.2-0.7, preferably in the range of 0.29-0.63;
β_{MMP9} is in the range of 0.00001-0.001, preferably in the range of 0.00018-0.00092;
β_{LG3BP} is in the range of (−0.002)-0.0002, preferably in the range of (−0.00021)-0.000022;
β_{THBS1} is in the range of (−0.00004)-0.000007, preferably in the range of (−0.000036)-0.0000068;
β_{FN1} is in the range of (−0.000004)-0.00001, preferably in the range of (−0.0000037)-0.0000011;
β_{LUM} is in the range of (−0.005)-0.03, preferably in the range of (−0.00055)-0.0028.

According to the invention, preferably for a low chance of BCR, a threshold value of the combined score value of below 50 or below 47.3, preferably in the range of 40.4-54.1 is selected, for a medium chance of BCR, a value of the combined score value between 50-75 or 47.3 to 71.1, preferably 40.4 to 79.5 is selected, and for a high chance of BCR, a threshold value of the combined score value of above 75 or above 71.1, preferably 62.6 to 79.5 is selected.

Further preferably, for a 90% sensitivity in the case of AP a threshold value of the combined score value of 36, preferably 30-42 is selected.

According to yet another preferred embodiment, the method includes:
a first step being performed by contacting the subject's serum, plasma or blood, preferably after dilution thereof, with at least one affinity reagent for each protein and detecting whether binding occurs between the respective protein and the at least one affinity reagent and using quantitative readout of the respective protein's concentration, allowing the calculation of the respective concentration in the original serum, plasma or blood, and wherein in this step either a sandwich enzyme linked immunosorbent assay specific to the respective protein preferably with visible readout is used, and/or a sandwich bead based antibody assay to the respective protein preferably with fluorescent readout.

The sandwich enzyme linked immunosorbent assay specific to the respective protein preferably with visible readout and/or the sandwich bead-based antibody assay to the respective protein preferably with fluorescent readout can be one obtained by using recombinant proteins of human THBS1, LUM, FN1, LG3BP, MMP9, respectively and mouse monoclonal antibodies generated through immunization of animal therewith.

The quantitative detection of the respective concentration may involve the determination of the concentration of such biomarkers relative to an external protein standard, involving the preparation of a reference standard curve by measuring defined concentrations of several, preferably 5-7 protein standards diluted in the same buffer as for the protein dilution to be measured in the same set of measurements of the samples.

Further embodiments of the invention are laid down in the dependent claims.

### BRIEF DESCRIPTION OF THE DRAWINGS

Preferred embodiments of the invention are described in the following with reference to the drawings, which are for the purpose of illustrating the present preferred embodiments of the invention and not for the purpose of limiting the same. In the drawings,

FIG. 1 shows Biochemical recurrence (BCR) free survival for CAPRA score (A) and Proposed Model (B).

FIG. 2 shows Association of CAPRA score (A) and Proposed Model (B) with adverse pathology (AP) features.

### DESCRIPTION OF PREFERRED EMBODIMENTS

### Study Population

The retrospective cohort included 557 men with localized PCa. All subjects underwent RP at the Martini Clinic (Hamburg, Germany) and had a clinical stage of cT<3 with or without staging lymphadenectomy. All blood samples were drawn prior RP, eight or more weeks after any prostatic manipulation (DRE, TRUS guided biopsy) and immediately processed and frozen. None of the patients had undergone any additional treatment.

The primary outcome was BCR after RP, defined as any postoperative PSA >0.2 ng/ml. Patients were censored at 5 years of follow-up. The secondary outcome was AP at RP, defined as either a pathological GG3 or greater, pathological stage of pT3a or greater, or positive pathological Node (pN1).

### Assay Methods

CE-IVD immunoassays were used for the quantification of CTSD and THBS1 (Proteomedix, Proclarix assays). Assays were performed according to the manufacturer's instructions. All other immunoassays were non-IVD immunoassays and composed of either commercially available components from R&D Systems (ATRN, ECM1, LG3BP, LRG1, LUM, MMP9, NCAM1, TIMP1, VEGF, ZAG) or reagents proprietary to Proteomedix (CFH, FN1, HYOU1, ICAM1, OLFM4, POSTN, VTN). The format used was either ELISA (CTSD, THBS1, CFH, FN1, VTN, POSTN) or Luminex (all other markers). Proprietary recombinant proteins (HYOU1, ICAM1, OLFM4) and commercially available recombinant proteins (all other markers) were used as reference for the calibration of the immunoassays.

### Statistical Methods

The proposed biomarker model for prognosis of patients with BCR was developed as follows: for all 20 markers univariate Cox proportional hazard (CoxPH) on BCR and General Linear Model (GLM) on AP was created. Markers regulated in the same direction (up or down) for BCR and AP were kept for further model building. Step Akaike Information Criteria (StepAIC) selection was then applied using CoxPH on BCR and GLM on AP. Finally, a multivariate CoxPH model was used to create the algorithm of the new proposed model.

The goodness-of-fit of the CoxPH model was assessed using the Schoenfeld's approach.

A nonsignificant result for this test indicates no deviation from the proportional hazard assumption, thus the proposed CoxPH model would be robust.

The best CoxPH model comprising FN1, LG3BP, LUM, MMP9, THBS1 and PSA together with GG was selected as the new proposed model.

The combined biomarker model value is preferably calculated using the following formula:

$$[ 1 - \frac{1}{1 + {e}^{( {β}_{0} + {β}_{1} * {x}_{1} + … + {β}_{k} * {x}_{k} )}} ] * 100$$

wherein βi are the regression coefficients as determined beforehand with an optimization, typically a maximization of the AIC in a CoxPH approach, using experimental data, β₀ being the correction factor based on the mean of the different variables, and wherein xᵢ is the measured concentration (ng/ml) of the respective protein in the original serum, plasma or blood and in case of GG it is the Gleason grade group (expressed as integer in the range of 1-5). The index therefore is 7.

For the calculation of the combined score value the regression coefficients are chosen as follows:

| | (coef) | lower .95 | upper .95 |
|---|---|---|---|
| Beta 0 | −0.9802507 | NA | NA |
| GG | 0.4628681 | 0.2906840 | 0.6350522 |
| PSA | 0.0104461 | −0.0109829 | 0.0318751 |
| MMP9 | 0.0005572 | 0.0001846 | 0.0009299 |
| LG3BP | −0.0000935 | −0.0002088 | 0.0000218 |
| THBS1 | −0.0000145 | −0.0000358 | 0.0000068 |
| FN1 | −0.0000013 | −0.0000037 | 0.0000011 |
| LUM | 0.0011091 | −0.0005526 | 0.0027708 |

In the above formula, the parameters are thus preferably chosen as follows:
β₀ is in the range of (−2)-0, preferably in the range of (−1.5)-(-0.5);
β_{tPSA} (total PSA) is in the range of 0-0.4, preferably in the range of 0.01-0.31;
β_{GG} in the range of 0.2-0.7, preferably in the range of 0.29-0.63;
β_{MMP9} is in the range of 0.00001-0.001, preferably in the range of 0.00018-0.00092;
β_{LG3BP} is in the range of (−0.002)-0.0002, preferably in the range of (−0.00021)-0.000022;
β_{THBS1} is in the range of (−0.00004)-0.000007, preferably in the range of (−0.000036)-0.0000068;
β_{FN1} is in the range of (−0.000004)-0.00001, preferably in the range of (−0.0000037)-0.0000011;
β_{LUM} in the range of (−0.005)-0.03, preferably in the range of (−0.00055)-0.0028.

For a low chance of BCR, preferably a threshold value of the combined score value of below 47.3, preferably 40.4-54.1 is selected. For a medium chance of BCR, in this case a value of the combined score value between 47.3 to 71.1, preferably 40.4 to 79.5 is selected. For a high chance of BCR, in this case a threshold value of the combined score value of above 71.1, preferably 62.6 to 79.5 is selected.

For a 90% sensitivity in the case of AP preferably a threshold value of the combined score value of 36, preferably 30-42 is selected.

The prognostic utility of the proposed model on BCR was assessed by using the Kaplan-Meier time-to-event approach. Results of the proposed model were compared to NCCN criteria [15] or CAPRA score [8]. For discriminative ability of AP at RP, the two-sided t-test p<0.05 was considered as statistically significant. All statistical analysis was performed using R statistical packages version 4.0.2 and GraphPad PRISM version 6.0.

### Results

### Biopsy Outcome

Patient characteristics are displayed in Table 1. Of the 557 men included in the study, the median (min-max) age was of 65 (44-78). The large majority of the patients had a low to intermediate risk of PCa based on NCCN criteria (87% of the population) or CAPRA score (89%). Among the 557 patients, 31% showed an AP event at RP. Fourteen percent of 5 patients had BCR within 5 years. The median follow-up time for those without BCR was 7.0 years (IQR 5.0, 7.4).

TABLE 1
Clinical characteristic of the patients

n (%)

General

| | | |
|---|---|---|
| All patients, n (%) | 557 | (100) |
| Median age at diagnosis, years (range) | 65 | (44-78) |

Biopsy characteristics

| | | | |
|---|---|---|---|
| ≤10 | ng/ml | 460 | (83) |
| 10-20 | ng/ml | 75 | (13) |
| >20 | ng/ml | 22 | (4) |

Grade Group

| | | |
|---|---|---|
| 1 | 257 | (46) |
| 2 | 169 | (30) |
| 3 | 76 | (14) |
| 4 | 38 | (7) |
| 5 | 17 | (3) |

Clinical Stage

| | | |
|---|---|---|
| cT1 | 474 | (85) |
| cT2 | 83 | (15) |

NCCN risk

| | | |
|---|---|---|
| low | 200 | (36) |
| intermediate | 282 | (51) |
| high | 75 | (13) |

CAPRA score

| | | |
|---|---|---|
| CAPRA 0 | 2 | (0.4) |
| CAPRA 1 | 86 | (15) |
| CAPRA 2 | 143 | (26) |
| CAPRA 3-5 | 269 | (48) |
| CAPRA 6-10 | 57 | (10) |

Surgical characteristics Grade Group

| | | |
|---|---|---|
| 1 | 85 | (15) |
| 2 | 385 | (69) |
| 3 | 76 | (14) |
| 4 | 5 | (1) |
| 5 | 6 | (1) |

pathological Stage

| | | |
|---|---|---|
| pT2 | 429 | (77) |
| pT3 | 128 | (23) |

Regional Lymph Nodes

| | | |
|---|---|---|
| N0 | 431 | (77) |
| N1 | 17 | (3) |
| NX | 109 | (20) |

Progression to agressiv PCa Progression to BCR

| | | |
|---|---|---|
| Events | 77 | (14) |
| Median years to follow up (range) ^{(a)} | 7.0 | (5.0-7.4) |

Progression to AP

| | | |
|---|---|---|
| GG > 2 | 84 | (15) |
| pT > 2 | 128 | (23) |
| N > 0 | 17 | (3) |
| Total ^{(b)} | 170 | (31) |

^{(a)} Follow up for men who had not experienced an event
^{(b)} multiple events for the same patient possible

### Proposed Model Building

Univariate CoxPH models on BCR and GLMs on AP are shown in Table 2A.

TABLE 2
Univariate analysis (A): Hazard ratio of Cox proportional hazards
regression (CoxPH) on Biochemical recurrence (BCR) after surgery
and odd ratios of General Linear Model (GLM) on Adverse Pathology
(AP). Multivariate analysis (B): CoxPH on BCR, the proposed model
is composed of Grade Group + PSA + LUM + FN1 + LG3BP + MMP9 + THBS1
A

| | Units | | CoxpH model on BCR | | Glm model on AP | |
|---|---|---|---|---|---|---|
| Marker | increase | | HR (95% CI) | p-value | OR (95% CI) | p-value |
| Age | 1 | year | 0.99 (0.96-1.02) | 0.390 | 1.07 (1.04-1.10) | <0.001 |
| Grade Group | 1 | unit | 1.60 (1.35-1.90) | <0.001 | 1.67 (1.38-1.99) | <0.001 |
| Prostate volume | 10 | ml | 0.95 (0.84-1.07) | 0.380 | 0.90 (0.81-1.01) | 0.064 |
| PSA | 1 | ng/ml | 1.03 (1.01-1.05) | 0.010 | 1.07 (1.03-1.10) | <0.001 |
| ATRN | 1 | μg/ml | 0.99 (0.96-1.02) | 0.515 | 1.01 (0.99-1.04) | 0.322 |
| CFH | 1 | μg/ml | 1.00 (1.00-1.01) | 0.432 | 1.00 (1.00-1.01) | 0.170 |
| CTSD | 100 | ng/ml | 0.94 (0.79-1.11) | 0.469 | 0.99 (0.86-1.14) | 0.869 |
| ECM1 | 100 | ng/ml | 0.96 (0.91-1.02) | 0.152 | 0.99 (0.94-1.04) | 0.667 |
| FN1 | 1 | μg/ml | 1.00 (1.00-1.00) | 0.210 | 1.00 (1.00-1.00) | 0.732 |
| LG3BP | 1 | μg/ml | 0.93 (0.83-1.04) | 0.195 | 0.96 (0.89-1.05) | 0.377 |
| HYOU1 | 100 | ng/ml | 1.75 (0.80-3.83) | 0.165 | 1.05 (0.53-2.10) | 0.883 |
| ICAM1 | 100 | ng/ml | 1.48 (0.83-2.65) | 0.182 | 1.51 (0.91-2.51) | 0.110 |
| LRG1 | 1 | μg/ml | 1.09 (0.97-1.22) | 0.135 | 1.12 (0.97-1.30) | 0.119 |
| LUM | 100 | ng/ml | 1.04 (0.89-1.23) | 0.601 | 1.09 (0.97-1.23) | 0.131 |
| MMP9 | 100 | ng/ml | 1.05 (1.02-1.09) | 0.002 | 1.00 (0.97-1.04) | 0.972 |
| NCAM1 | 100 | ng/ml | 0.91 (0.70-1.18) | 0.469 | 0.99 (0.80-1.23) | 0.923 |
| OLFM4 | 100 | ng/ml | 1.63 (1.01-2.62) | 0.044 | 0.82 (0.49-1.36) | 0.436 |
| POSTN | 100 | ng/ml | 0.79 (0.57-1.11) | 0.172 | 1.02 (0.93-1.11) | 0.728 |
| THBS1 | 1 | μg/ml | 0.99 (0.97-1.01) | 0.319 | 0.99 (0.98-1.01) | 0.579 |
| TIMP1 | 100 | ng/ml | 1.09 (0.95-1.26) | 0.217 | 0.99 (0.87-1.15) | 0.954 |
| VEGF | 1 | μg/ml | 1.05 (0.27-4.08) | 0.949 | 1.01 (0.32-3.16) | 0.990 |
| VTN | 1 | μg/ml | 1.02 (0.99-1.04) | 0.170 | 1.00 (0.98-1.02) | 0.966 |
| ZAG | 1 | μg/ml | 1.05 (0.96-1.16) | 0.267 | 1.02 (0.93-1.11) | 0.715 |

| B | |
|---|---|
| | CoxpH Model for BCR |

| | Units | | | | concordance |
|---|---|---|---|---|---|
| Model | increase | | HR (95% CI) | p-value | coefficient |
| CAPRA | 1 | unit | 1.36 (1.21-1.53) | <0.001 | 0.643 |
| Grade Group | 1 | unit | 1.60 (1.35-1.90) | <0.001 | 0.664 |
| Grade Group + PSA | 5 | units | 1.25 (1.16-1.35) | <0.001 | 0.676 |
| Proposed model | 5 | units | 1.28 (1.19-1.38) | <0.001 | 0.715 |

Hazard Ratio (HR) and Odd Ratios (OR) comparison ruled out age, ATRN, OLFM4, POSTN and TIMP1 for further model building. Stepwise selection applied for CoxPH on BCR and for GLM on AP, yielded a 9-plex model for BCR (GG, PSA, ECM1, FN1, LG3BP, LUM, MMP9, THBS1 and VTN) and 5-plex model for AP (GG; prostate volume, PSA, LG3BP and LUM). Out of those 10 different variables, the performance of 20 different multivariate CoxPH models combining 5 to 7 variables were tested for discrimination of low versus intermediate and high risk of BCR. Acceptable low risk fraction of BCR was set to be below 5% after 5 years. Finally, the best CoxPH model comprising FN1, LG3BP, LUM, MMP9, THBS1 and PSA together with GG was selected as the new proposed model.

Multivariate analysis of the proposed model for CoxPH on BCR is shown in Table 2B.

The proposed model is significantly associated to BCR (HR 1.28 per 5 units score, 95% CI 1.19-1.38, p<0.001). The addition of PSA to the GG and in a second step of the 5 serum markers to GG+PSA improved the prediction of BCR by increasing the c-index respectively by 0.051 and 0.039. The Schoenfeld's approach for testing the goodness-of-fit of the CoxPH model showed no difference between the observed covariate and the expected given risk set at that time. The test was not statistically significant for each of the covariates (p>0.07) and for the proposed model (p=0.76, supplementary data). Therefore, we can assume no deviations from the proportional hazard assumptions.

### Kaplan-Meier Analysis on BCR Prediction

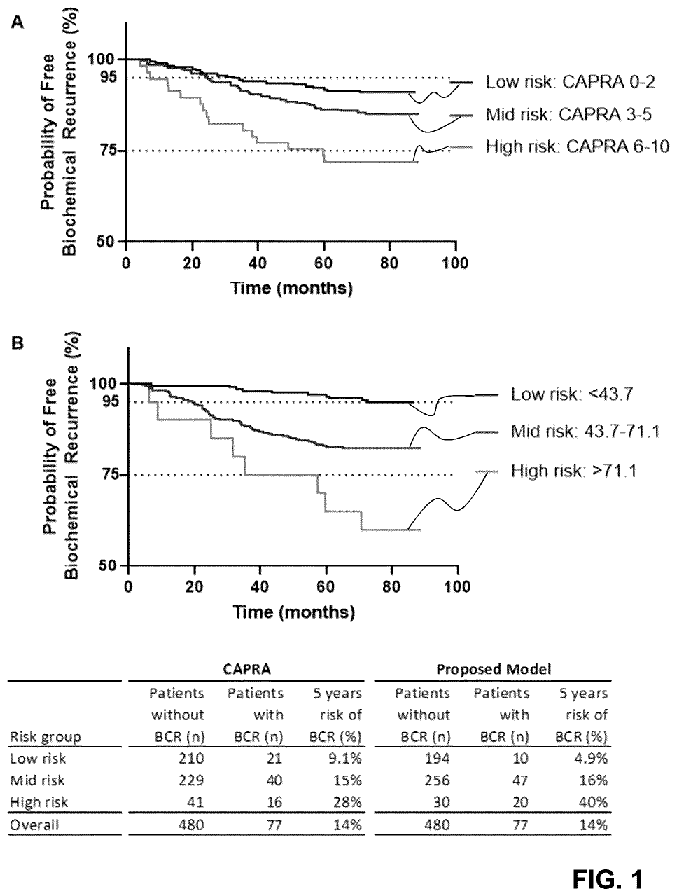

The Kaplan-Meier analysis of freedom from BCR is shown in FIG. 1. Thresholds for the proposed model were identified in order to stratify the population in no BCR (<37.8), low risk (<47.3), intermediate risk (47.3-71.1) and high risk (>71.1) of BCR. For the proposed model, definition of low risk of BCR after 5 year was set to be lower than 5%, and higher than 40% for high risk of BCR.

As a result, the Kaplan-Meier analysis of the overall cohort showed that the proposed model has a better prediction of low-risk BCR after RP compared to CAPRA (respectively 4.9% vs. 9.1% chance of BCR, for n=194 and n=210 patients). Those results show the superior ability of the proposed model to discriminate patients with the low risk of BCR. These findings were similar when applying the proposed model in cohorts with pre-defined low risk of BCR by selecting patients with CAPRA<2 (n=231), GG<2 (n=257) or NCCN=low risk (n=200).

Results are shown in Table 3.

TABLE 3
Performance of the Proposed Model for Biochemical recurrence (BCR) free
survival in CAPRA 0-2, NCCN low and Grade Group 1 patient population.

| | | CAPRA 0-2 | NCCN Low | Grade Group 1 |
|---|---|---|---|---|
| Risk of | Threshold from | Patients ^{(a)} | Patients | Patients |
| BCR | Proposed Model | (n, % BCR risk) | (n, % BCR risk) | (n, % BCR risk) |
| Low Risk | <43.7 | n = 138, 3.6% BCR | n = 142, 4.9% BCR | n = 170, 5.3% BCR |
| Mid Risk | 43.7-71.1 | n = 91, 17% BCR | n = 58, 7% BCR | n = 58, 7% BCR |
| High Risk | >71.1 | n = 2, 50% BCR | none | none |
| Overall | n/a | n = 231, 9% BCR | n = 200, 5.5% BCR | n = 257, 7% BCR |

^{(a)} n = 2 patients with CAPRA = 0

Here, the risk of BCR using the low-risk cutoff of the proposed model (<37.8) was below 5.5% (n>138 patients) in all three subgroups and thus lower when compared to CAPRA=0-2 (9%), GG<2 (7%) and NCCN=low-risk (6%) subsets.

### Discrimination of Adverse Pathology

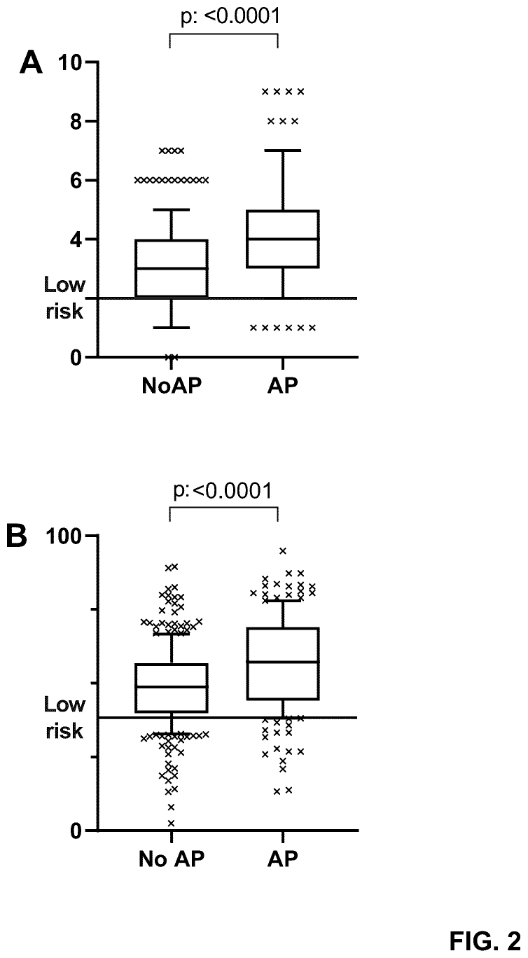

When applying a threshold <36, the proposed model is significantly associated with AP at RP (p<0.001; FIG. 2) as well as with the three single AP events (p<0.001 for GG>2, pT>2 and pN1; supplementary data). The clinical performance for the prediction AP was not superior, but only equivalent to CAPRA (supplementary data): when applying a threshold CAPRA<2 and a cutoff of <36 for the proposed model, the sensitivity and specificity between the two models turned out to be not significantly different (p-values of 0.090 when comparing sensitivities and 0.159 when comparing specificities).

### Discussion

The ability to assess prognosis of PCa is critical for the management of men undergoing a RP. The difficulty of the prediction of PCa is enhanced by the variety of adverse outcome linked to PCa progression: BCR, AP, metastasis or death. The ideal prognostic model would need to cover all these aspects in order to help on the decision making for possible post-operative treatments. The current stratification of the risk in clinical practice remains fairly poor. Various free nomograms (i.e. CAPRA, d'Amico score) have been developed based on pathological outcome. Commercially available tests like the CCP-score, a tissue based genomic test of 31 call cycle progression genes or the GPS-score, a test based on the RNA expression of 17 genes, could also stratify the risk of PCa progression, as it was shown in multiple studies for CAPRA, CCP or GPS. However, the difficulty to identify one logical threshold, with which to guide treatment across different cohorts remains challenging.

In this study we evaluated the prognostic usability of a new proposed model for the assessment of BCR after RP, and AP at RP. The performance of the proposed model was compared to the CAPRA. All patients from the study population (n=557) had a clinical stage below 3.

As expected, the prognostic capability of CAPRA for BCR was limited in the cohort. The large amount of low-risk patients (CAPRA 0-2, 9.1% BCR, n=210) and very low number of patients without BCR (CAPRA=0, n=2) makes it of limited use for safe treatment guidance of the patients.

Here we first developed a model with protein biomarkers originally discovered in the context of PTEN-mutation using mouse model. The multivariable model is combining THBS1, LUM, FN1, MMP9, LG3BP together with PSA and clinical GG. Even though not all markers were significantly associated with BCR or AP in a univariable analysis, the proposed model could significantly (p<0.001) discriminate patients with AP events at RP and was a significant predictor of BCR (HR 1.28 per 5 units score, 95% CI 1.19-1.38, p<0.001). Those findings are supported with the analysis of the c-index, which increases when adding the four biomarkers to the PSA and GG.

The proposed model shows a superior prediction of BCR after RP compared to CAPRA. It could predict no risk of BCR for 12.6% of the population, where CAPRA predicted less than 0.1% with CAPRA=0. It could also predict 4.9% recurrence if applying a low-risk threshold of below 37.8 (n=194) compared to 9.1% for low-risk CAPRA=0-2 (n=210). A risk of less than 5% could be considered as fairly low, putting patients at an appreciable risk of BCR after RP.

Among the different low-risk patient population defined as CAPRA=0-2, NCCN=low and GG<2, the proposed model was with less than 5.3% risk of BCR again slightly superior to CAPRA score (9% risk of BCR), NCCN (6%) and GG (7%).

Only 14% patients had a BCR within 5 years. This is due to the selection criteria excluding patients undergoing neoadjuvant and adjuvant treatment as well as selecting cT <3 patients. Nevertheless, the cohort used for this study can be considered as representative of a low-risk patient population, where risk stratification remains especially challenging. The cohort is comparable to the ones used in other studies, also assessing various models on BCR

risk after RP [23].

The present study has some limitations that should be noted. The main limitation is that the proposed model was trained on a single retrospective cohort, restricted to one single centre, with mainly Caucasian men. A generalization of the model to more diverse populations is therefore limited. Additionally, another limitation is the lack of proper validation of the model.

Even if the goodness-of-fit of the CoxPH model was assessed using the Schoenfeld's approach, performance of the proposed model and its selected threshold cannot be extrapolated when applied to another independent cohort. Finally, we could show that the proposed model was significantly associated only with BCR and AP. The association to other relevant prognostic endpoints (i.e. death or metastasis) could not be assessed within this cohort.

In conclusion the proposed model improved the clinical stratification of BCR-risk and AP of men undergoing prostatectomy. The model could potentially better guide treatment selection, but validation studies should be performed in independent cohorts in order to validate the model.

1. A method for collecting information about the health status of a subject involving the quantitative detection, in serum, plasma or blood of the subject, of the concentration of at least four of the systems selected from the group consisting of: THBS1, LUM, FN1, LG3BP, MMP9, as well as PSA.

2. Method according to claim 1, involving the quantitative detection, in serum, plasma or blood of the subject, of the concentration of each of THBS1, LUM, FN1, LG3BP, MMP9, as well as PSA.

3. Method according to claim 1, wherein the method includes
a first step being performed by contacting the subject's serum, plasma or blood, preferably after dilution thereof, with at least one, or two, affinity reagent for each protein and detecting whether binding occurs between the respective protein and the at least one affinity reagent and using quantitative readout of the respective protein's concentration, allowing the calculation of the respective concentration in the original serum, plasma or blood;
a second step of calculating, based on all the protein concentrations as well as the PSA concentration determined in the first step, a combined score value.

4. Method according to claim 3, wherein after the second step in a third step the risk of a Biochemical recurrence (BCR) after surgery of prostate cancer and/or of adverse pathology (AP) of the subject is determined based on the combined score value as determined in the second step, wherein surpassing a corresponding threshold value of the combined score value is taken as positive prostate cancer Biochemical recurrence after surgery and/or as necessity of prostatectomy.

5. Method according to claim 1, wherein the combined score value is calculated based on the measured concentrations x_{tPSA}, x_{MMP9}, x_{LG3BP}, x_{THBS1}, x_{FN1}, x_{LUM} and the Gleason grade (GG) of at least one preceding biopsy expressed as integer in the range of 1-5 using the following formula:
$$[ 1 - \frac{1}{1 + {e}^{( {β}_{0} + {β}_{1} * {x}_{1} + … + {β}_{k} * {x}_{k} )}} ] * 100$$
and β₀; β_{tPSA}; β_{GG}; β_{MMP9}; β_{LG3BP}; β_{THBS1}; β_{FN1}; β_{LUM}.

6. Method according to claim 5, wherein
β₀ is in the range of (−2)-0, preferably or in the range of (−1.5)-(−0.5); and/or
β_{tPSA} is in the range of 0-0.4, preferably or in the range of 0.01-0.31; and/or
β_{GG} in the range of 0.2-0.7, preferably or in the range of 0.29-0.63; and/or
β_{MMP9} is in the range of 0.00001-0.001, preferably or in the range of 0.00018-0.00092; and/or
β_{LG3BP} is in the range of (−0.002)-0.0002, or preferably-in the range of (−0.00021)-0.000022; and/or
β_{THBS1} is in the range of (−0.00004)-0.000007, or preferably-in the range of (−0.000036)-0.0000068; and/or
β_{FN1} is in the range of (−0.000004)-0.00001, or preferably in the range of (−0.0000037)-0.0000011; and/or
β_{LUM} is in the range of (−0.005)-0.03, or preferably-in the range of (−0.00055)-0.0028.

7. Method according to claim 6, wherein
for a low chance of BCR, a threshold value of the combined score value of below 50 or below 47.3, or in the range of 40.4-54.1 is selected,
for a medium chance of BCR, a value of the combined score value between 50-75 or 47.3 to 71.1, or 40.4 to 79.5 is selected
for a high chance of BCR, a threshold value of the combined score value of above 75 or above 71.1, or 62.6 to 79.5 is selected.

8. Method according to claim 6, wherein for a 90% sensitivity in the case of AP a threshold value of the combined score value of 36, or 30-42 is selected.

9. Method according to claim 1, wherein the method includes
a first step being performed by contacting the subject's serum, plasma or blood, preferably after dilution thereof, with at least one affinity reagent for each protein and detecting whether binding occurs between the respective protein and the at least one affinity reagent and using quantitative readout of the respective protein's concentration, allowing the calculation of the respective concentration in the original serum, plasma or blood, and wherein in this step either a sandwich enzyme linked immunosorbent assay specific to the respective protein preferably with visible readout can be used, and/or a sandwich bead-based antibody assay to the respective protein with fluorescent readout.

10. Method according to claim 9, wherein the sandwich enzyme linked immunosorbent assay specific to the respective protein with visible readout and/or the sandwich bead-based antibody assay to the respective protein preferably with fluorescent readout is one obtained by using recombinant proteins of human THBS1, LUM, FN1, LG3BP, MMP9, respectively and animal monoclonal antibodies generated through immunization of mice therewith.

11. Method according to claim 1, wherein the quantitative detection of the respective concentration involves the determination of the concentration of such biomarkers relative to an external protein standard, involving the preparation of a reference standard curve by measuring defined concentrations of several, or 5-7 protein standards diluted in the same buffer as for the protein dilution to be measured in the same set of measurements of the samples.

12. Method according to claim 1, wherein further the Gleason grade (GG) of at least one preceding biopsy is taken account of, expressed as integer in the range of 1-5.
